(19) United States
(12) Patent Application Publication
(10) Pub. No.: US 2005/0261282 A1
(43) Pub. Date: Nov. 24, 2005

(54) Treatment of basal ganglia-related movement disorders with 2,3-benzodiazepines

(75) Inventors: Alan Crossman, Manchester (GB); Michael Hill, Manchester (GB); Jonathan Brotchie, Toronto (CA)

Correspondence Address: GATES & COOPER LLP;HOWARD HUGHES CENTER, 6701 CENTER DRIVE WEST, SUITE 1050, LOS ANGELES, CA, 90045, US

(73) Assignee: Motac Neuroscience Limited Williams House, Manchester (GB)

(21) Appl. No.: 10/527,271

(22) PCT Filed: Sep. 11, 2003
(86) PCT No.: PCT/GB03/03951

§ 371 Date: Mar. 8, 2005

(30) Foreign Application Priority Data
Sep. 13, 2002 (GB) 0221248.8
Feb. 25, 2003 (GB) 0304227.2

(51) Int. Cl. A61K 31/5513

(52) U.S. Cl. 514/221

(57) ABSTRACT

The present invention relates to compounds, related to 2,3 benzodiazepines, of general formula (I), such as Tofisopam, Girisopam or Nerisopam, for use in the treatment of dyskinesia. The dyskinesia may arise as a side-effect of a therapy for pakinsonism.

The present invention relates to the treatment of basal ganglia-related movement disorders and particularly dyskinesias.

Dyskinesias are abnormal involuntary movement disorders. The abnormal movements may manifest as chorea (involuntary, rapid, irregular, jerky movements that may affect the face, arms, legs, or trunk), ballism (involuntary movements similar to chorea but of a more violent and forceful nature), dystonia (sustained muscle contractions, usually producing twisting and repetitive movements or abnormal postures or positions) or athetosis (repetitive involuntary, slow, sinuous, writhing movements, which are especially severe in the hands).

Movement and other disorders due to dysfunction of the basal ganglia and related brain structures are of major socio-economic importance. Such disorders can occur as a consequence of inherited or acquired disease, idiopathic neurodegeneration or they may be iatrogenic. The spectrum of disorders is very diverse, ranging from those associated with poverty of movement (akinesia, hypokinesia, bradykinesia) and hypertonia (e.g. Parkinson's disease, some forms of dystonia) to the involuntary movement disorders (hyperkinesias or dyskinesias e.g. Huntington's disease, levodopa-induced dyskinesia, ballism, some forms of dystonia).

Knowledge of the pathophysiological mechanisms that underlie some of these disorders makes it likely that similar mechanisms mediate disorders characterised by either hyperkinesias or dyskinesias. It is to be expected, therefore, that treatments that are effective in one form of dyskinesia may be beneficial in dyskinesias of different aetiology.

One common way in which dyskinesias arise is as a side-effect of dopamine replacement therapy for parkinsonism or other basal ganglia-related movement disorders. Parkinsonism is a syndrome of symptoms characterised by slowness of movement (bradykinesia), rigidity and/or tremor. Parkinsonian symptoms are seen in a variety of conditions, most commonly in idiopathic parkinsonism (i.e. Parkinson's disease) but also following treatment of schizophrenia, exposure to toxins/drugs and head injury. In Parkinson's disease the primary pathology is degeneration of dopaminergic neurons of the substantia nigra, pars compacta.

The most widely used symptomatic treatments for parkinsonism use dopamine-replacing agents (e.g. L-DOPA and dopamine receptor agonists). These do, however, have limitations, especially following long-term treatment. Problems can include a “wearing-off” of the anti-parkinsonian efficacy of the treatment and in particular the appearance of a range of side-effects. These side-effects may manifest as dyskinesias such as chorea and dystonia. Dyskinesia can be seen either when the patient is undergoing dopamine-replacement therapy (in the case of chorea and/or dystonia) or even when off therapy (when dystonia is prevalent). Ultimately, these side-effects severely limit the usefulness of dopaminergic treatments.

Another common cause of dyslinesias is the treatment of psychosis with neuroleptic drugs—this is known as tardive dyslinesia.

Dyskinesia also occurs in many other conditions including:
Huntington's disease
idiopathic dystonia
Tourette syndrome
“off” dystonia in parkinsonism
ballism
senile chorea

Many attempts have been made to develop agents that will prevent the development of, and/or treat, dyskinesias although such attempts have met with limited success. There is, therefore, a need to develop ways by which dyskinesias may be treated.

The present invention relates to the treatment of dyskinesias using a compound with the general formula (I) 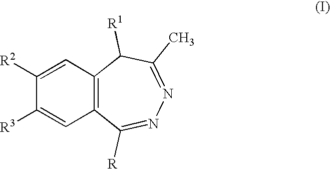
wherein R is an aryl group, such as phenyl or benzyl, which is optionally substituted with a C₁₋₆ alkyl, C₁₋₆ alkoxy, halogen, hydroxyl, amino, nitro, amido, nitrile or a carboxyl group;
R¹ is C₁₋₆ alkyl or hydrogen;
R² is C₁₋₆ alkoxy, hydrogen, hydroxyl, or halogen; and
R³ is C₁₋₆ alkoxy, hydrogen, hydroxyl, or halogen,

Preferably R is selected from one of the following groups:

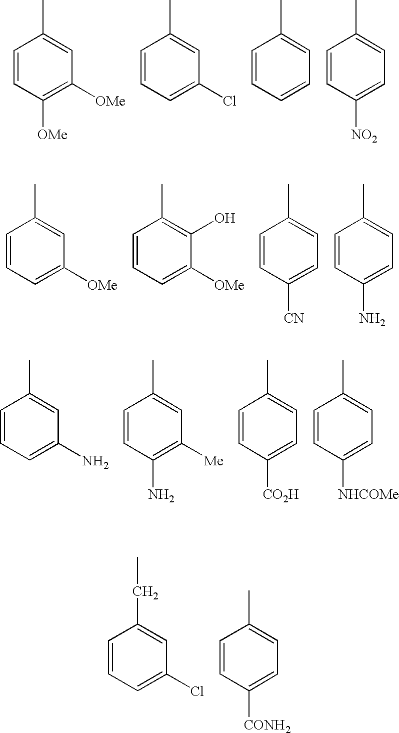

When R¹ is an alkyl group, it is preferred that R¹ is C₁₋₃ alkyl with C₂ alkyl(ethyl) being most preferred.

When R² is an alkoxy group, it is preferred that R² is C₁₋₃ alkoxy with C₁ alkoxy (methoxy) being most preferred.

When R³ is an alkoxy group, it is preferred that R³ is C₁₋₃ alkoxy with C₁ alkoxy (methoxy) being most preferred.

It is preferred that the compound of formula (I) is selected from the group of Tofisopam, Girisopam and Nerisopam, which are as follows:

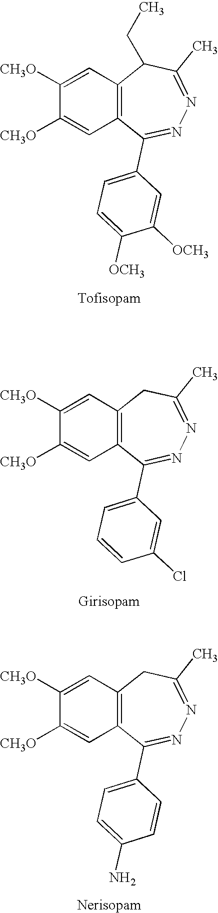

Most preferably the compound of formula (I) is Tofisopam.

It will be appreciated that different stereoisomers of compounds of formula (I) may exist and such stereoisomers may be be employed to treat dyskinesia according to the invention. For instance it will be appreciated that compounds such as Tofisopam have a chiral centre. Accordingly Tofisopam, and other compounds of formula (I) that possess a chiral centre, can exist as R and S enantiomers. The present invention encompasses the use of such compounds as a racemic mixture or the use of separated R or S enantiomers.

According to a first aspect of the present invention, there is provided a use of a compound of general formula (I) for the manufacture of a medicament for the treatment of dyskinesia.

According to a second aspect of the present invention, there is provided a composition for use in the treatment of dyskinesia comprising a therapeutically effective amount of a compound of general formula (I) and a pharmaceutically acceptable vehicle.

According to a third aspect of the present invention, there is provided a method for the treatment of dyskinesia comprising administering to a person or animal in need of said treatment a therapeutically effective amount of a compound of general formula (I).

According to a fourth aspect of the present invention, there is provided a use of an agent which modulates the activity of receptors with ligands of general formula (I) for the manufacture of a medicament for the treatment of dyskinesia.

By “receptors” we mean receptors located on the cell bodies and terminals of neurons located within the striatum, the internal and external segments of the globus pallidus and the substantia nigra in the brain and thereby induce a neuronal signal which reduces dyskinesia and for which molecules of general formula (I) act as ligands.

By “dyskinesia” we mean abnormal involuntary movements that are associated with disorders of brain regions known as the basal ganglia. The dyskinesia may be a “levodopa-induced dyskinesia” that arises as a complication of the treatment of Parkinson's disease (the most common basal ganglia disease). Dyslinesia can physically manifest in two forms, chorea and dystonia. Chorea consists of involuntary, continuous, purposeless, abrupt, rapid, brief, unsustained and irregular movements that flow from one part of the body to another. Dystonia refers to sustained muscle contractions that cause twisting and repetitive movements or abnormal postures.

Dyskinesias may be distinguished from ataxia or catalepsy. Ataxia is usually associated with disorders of a part of the brain called the cerebellum, or its connections. It is characterised by poor motor coordination. There is a staggering gait (walk) and slurred speech, which may make the person appear “drunk”. Catalepsy is, again, a different condition that is impossible to confuse with dyskinesias. It is usually associated with psychotic disorders. It is characterized by inactivity, decreased responsiveness to stimuli, and a tendency to maintain an immobile posture. The limbs tend to remain in whatever position they are placed. Thus, the terms dyskinesia (including chorea and dystonia), ataxia and catalepsy refer to distinct and separate disorders. They have different physical manifestations and different causes.

The present invention is based upon research conducted by the inventors relating to the activity of compounds of general formula I. To their surprise they found that such compounds have efficacy for reducing dyskinesias. This lead them to realise that several classes of agent may be used according to the fourth aspect of the invention. These include:
(i) exogenous 2,3 benzodiazepine receptor ligands;
(ii) compounds which enhance synthesis of endogenous 2,3 benzodiazepine receptor ligands;
(iii) compounds which enhance release of endogenous 2,3 benzodiazepine receptor ligands;
(v) compounds which block the rate of inactivation or metabolism of endogenous 2,3 benzodiazepine receptor agonists; and
(vi) compounds which promote/increase 2,3 benzodiazepine receptor expression and/or transcription.

The invention is based upon our studies relating to the neural mechanisms underlying movement disorders. Although we do not wish to be bound by any hypothesis, we believe that movement disorders involve abnormal activity of basal ganglia output pathways and in many cases this is brought about by abnormal function of striatal efferent pathways. These consist of a “direct” pathway to the medial or internal segment of the globus pallidus and the pars reticulata of the substantia nigra and a “indirect” pathway to the lateral or external segment of the globus pallidus. One of the pathophysiological hallmarks of dyskinesia is overactivity of the direct striatal output pathway. Conversely, in Parkinson's disease the direct striatal output pathway is underactive and the indirect striatal output pathway is overactive. We believe compounds of general formula (I) bind to receptors located on the terminals of the striatal output neurons that project to the internal and external segments of the globus pallidus and the pars reticulata of the substantia nigra and thereby induce a neuronal signal which reduces dyskinesia.

WO 99/06408 and WO 01/04122 disclose molecules that have a core 2, 3 benzodiazepine tricyclic structure with superficial similarity to the compounds of general formula (I). The prior art speculates that such compounds may be used in the treatment of a variety of medical conditions (e.g. Parkinson's disease). However, the compounds used according to the invention are distinguished over this prior art in that the rings have different substituents (in particular at R, R² and R³). Furthermore, the compounds of general formula (I) have surprising utility for treating dyskinesias, that is to say excessive involuntary movements, and not Parkinson's disease per se, which is characterised by a poverty of movement.

The present inventors conducted experiments with the molecule Tofisopam that led them to realise that compounds of general formula (1) are highly effective for the treatment of dyskinesias. For instance, it was found that dyskinesias (e.g. chorea and dystonia) do not develop, or are at least reduced, when the compounds are given to subjects on dopamine-replacement therapy for the treatment of a movement disorder.

The compounds (and compositions or medicaments containing them) may be used to treat many types of dyskinesia For instance the compounds may be used to treat dyskinesia associated with Huntington's disease, idiopathic torsion dystonia, tardive dyskinesia or off-dystonia in Parkinson's disease and most particularly for dyskinesia associated with movement disorders such as parkinsonism (e.g. idiopathic Parkinson's disease, post-encephalitic parkinsonism or parkinsonism resulting from head injury), treatment of schizophrenia, drug intoxication, manganese poisoning and the like.

The compounds may also be used in the treatment of dyskinesias that manifest as hyperkinetic activity (e.g. Tourette's syndrome or attention deficit hyperactivity (ADHD)).

The compounds are also useful for treatment of dyskinesias that arise as a side-effect of other therapeutic agents. For instance, the compounds are useful for the treatment of dyskinesia associated with ropinirole, pramipexole, cabergoline, bromcriptine, lisuride, pergolide, L-DOPA or apomorphine treatment. The compounds are preferably used for the treatment of dyskinesia associated with L-DOPA or apomorphine treatment.

Levodopa is an aromatic amino acid. The chemical name of levodopa or L-DOPA is (−)-L-α-amino-#-(3,4-dihydroxybenzene)propanoic acid. L-DOPA has the molecular formula C₉H₁₁NO₄ and a molecular weight of 197.2. Chemically, levodopa is (−)-3-(3,4-dihydroxy-phenyl)-L-alanine. It is a colorless, crystalline compound, slightly soluble in water and insoluble in alcohol. L-DOPA has the following structural formula:

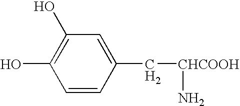

Because L-DOPA is an amino acid, it is commonly administered to patients in combination with carbidopa for the treatment of Parkinson's disease and syndrome. The chemical name for carbidopa is (−)-L-α-hydrazino-α-methyl-β-(3,4-dihydroxybenzene)propanoic acid monohydrate. Carbidopa has the empirical formula C₁₀H₁₄N₂O₄.H₂O and a molecular weight of 244.3. Anhydrous carbidopa has a molecular weight of 226.3. Sinemet® is a combination of carbidopa and levodopa for the treatment of Parkinson's disease and syndrome. Sinemet® is described in U.S. Pat. Nos. 4,832,957 and 4,900,755, the contents of which are herein incorporated by reference. The structural formula of carbidopa is:

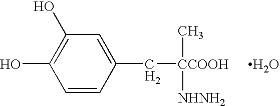

In addition, the compounds according to the invention are useful for the treatment of dyskinesias associated with ropinirole treatment. Ropinirole is a non-ergoline dopamine agonist sold under the trademark Requip®. Ropinirole is the hydrochloride salt of 4-[2-(dipropylamino)ethyl]-1,3-dihydro-2H-indol-2-one monohydrochloride and has an empirical formula of C₁₆H₂₄N₂O.HCl. The molecular weight of ropinirole is 296.84 (260.38 as the free base). Ropinirole is described in U.S. Pat. Nos. 4,452,808 and 4,824,860, the contents of which are hereby incorporated by reference. The structural formula of ropinirole is:

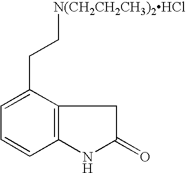

The compounds according to the invention are also useful for the treatment of dyskinesias associated with pramipexole treatment. The chemical name of pramipexole is (S)-2-amino-4,5,6,7-tetra-hydro-6-(propylamino)benzothiazole dihydrochloride mono-hydrate. Pramipexole dihydrochloride is sold under the trademark Mirapex®. Pramipexole dihydrochloride has the empirical formula C₁₀H₁₇N₃S.2HCl.H₂O and a molecular weight of 302.27. The synthesis of pramipexole is described in U.S. Pat. Nos. 4,843,086 and 4,886,812, the contents of which are herein incorporated by reference. The structural formula of pramipexole dihydrochloride is:

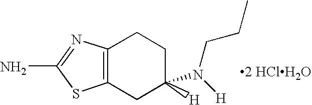

The compounds may also be used for the treatment of dyskinesias associated with cabergoline treatment. The chemical name for cabergoline is 1-adamantanamine hydrochloride. It has a molecular weight of 187.71 and a molecular formula of C₁₀H₁₈NCl. The structural formula of cabergoline is:

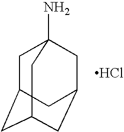

The compounds may also be used for the treatment of dyskinesias associated with bromocriptine treatment. Bromocriptine mesylate is sold under the trademark Parlodel®. The chemical name for bromocriptine mesylate is Ergotaman-3′,6′,18-trione, 2-bromo-12′-hydroxy-2′-(1-methylethyl)-5′-(2-methylpropyl)-, (5′a)-monomethanesulfonate. The molecular weight of bromocriptine mesylate is 750.70 and it has an empirical formula of C₃₂H₄₀BrN₅O₅.CH₄SO₃. The structural formula of bromocriptine mesylate is:

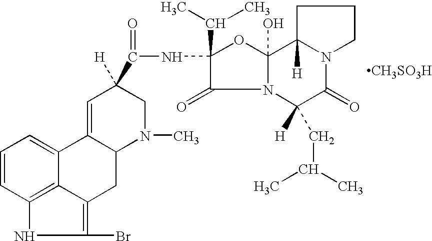

The compounds may also be used for the treatment of dyskinesias associated with lisuride treatment. The chemical name for lisuride is R(+)-N′-[(8α)-9,10-Didehydro-6-methylergolin-8-yl]-N,N-diethylurea hydrogen maleate. Lisuride has a molecular weight of 338.45 and the empirical formula C₂₀H₂₆N₄O. The structural formula of lisuride is:

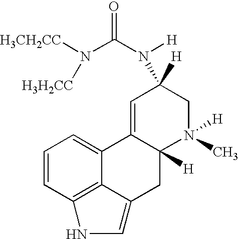

The compounds may also be used for the treatment of dyskinesias associated with pergolide treatment. The chemical name of pergolide mesylate is 8β-[(Methylthio)methyl]-6-propylergoline monomethanesulfonate. Pergolide mesylate is sold under the trademark Permax®. Permax has the empirical formula C₁₉H₂₆N₂S.CH₄O₃S and a molecular weight of 410.59. The synthesis of pergolide mesylate is described in U.S. Pat. Nos. 4,797,405 and 5,114,948, the contents of which are herein incorporated by reference. The structural formula of pergolide mesylate is:

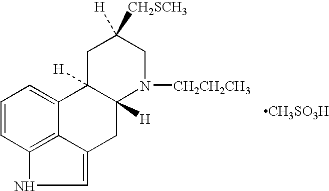

The compounds may also be used for the treatment of dyskinesias associated with apomorphine treatment. Apomorphine has the empirical formula C₁₇H₁₇NO₂ and a molecular weight of 267.33. The structural formula of apomorphine is:

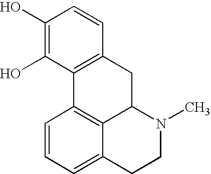

The compounds are particularly useful for treating dyskinesia caused by agents used to treat movement disorders such as parkinsonism. In this respect a preferred use of the compounds is in the treatment of dyskinetic side-effects associated with L-DOPA, apomorphine or other dopamine agonist therapy for parkinsonism.

The compounds may be used to treat existing dyslinesias but may also be used when prophylactic treatment is considered medically necessary. For instance, when it is considered necessary to initiate L-DOPA therapy and it is feared that dyskinesias may develop.

The compounds may be used to treat dyskinesia as a monotherapy (i.e. use of the compound alone) or they may be used as an adjunct to other therapeutic agents. For instance, the compounds may be co-administered with therapeutic agents to prevent dyslinetic side-effects caused by such therapeutic agents (e.g. as an adjunct to L-DOPA or apomorphine given to treat parkinsonian patients) or alternatively the compounds may be given in combination with other treatments which also reduce dyskinesia (e.g. μ-opioid receptor antagonists, α₂-adrenoreceptor-antagonists, cannabinoid CB₁-antagonists, NMDA receptor-antagonists, cholinergic receptor-antagonists, histamine H3-receptor agonists, and globus pallidus/subthalamic nucleus lesion/deep brain stimulation).

In preferred embodiments of the invention the compound of general formula (1) or agents according to the fourth aspect of the invention may be combined with therapeutic agents such as:
(a) therapeutic agents used in the treatment of parlinsonism, including Parkinson's disease (e.g. L-DOPA, Chloro-APB, apomorphine, ropinirole, pramipexole, cabergoline, bromcriptine, lisuride or pergolide)
(b) other therapeutic agents used in the treatment of dyskinesia (e.g. non selective, δ or μ-opioid receptor antagonists, α₂-adrenoreceptor-antagonists, cannabinoid CB₁-antagonists, Histamine H3 agonists, mGLuR antagonists NMDA receptor-antagonists, Gpi lesion/deep brain stimulation).
(c) therapeutic agents used as neuroleptics for the treatment of schizophrenia, psychosis and the like (e.g. agents with dopamine receptor antagonist properties, haloperidol clozapine, fluphenazine and sulpiride).

The compounds may also be used as an adjunct or in combination with known therapies. For instance, we have found that the combination of L-DOPA with compounds according to the invention results in movement disorders such as Parkinson's disease being treated with significantly reduced dyskinetic side-effects.

The compounds may also be used in combination with a known neuroleptic to treat patients suffering from tardive dyskinesia. The term neuroleptic refers to the effects on cognition and behavior of antipsychotic drugs that reduce confusion, delusions, hallucinations, and psychomotor agitation in patients with psychoses. There is a naturally occurring chemical, a neurotransmitter, in the brain called dopamine. Dopamine is the chemical messenger in the brain mainly involved with thinking, emotions, behavior and perception. In some illnesses, dopamine may be overactive and upsets the normal balance of chemicals in the brain. This excess dopamine helps to produce some of the symptoms of the illness. The main effect that these drugs have is to block some dopamine receptors in the brain, reducing the effect of having too much dopamine and correcting the imbalance. This reduces the symptoms caused by having too much dopamine.

Neuroleptic drugs are a class of antipsychotics. Examples of neuroleptic compounds include: haloperidol (Haldol), chlorpromazine (Thorazine), thioridazine (Mellaril), risperidone (Risperdal), quetiapine (Seroquel), olanzapine (Zyprexa), clozapine (Clozaril), amisulpride (Solian), sertindole (Serdolect), zotepine (Zoleptil), Thiothixene (Navane), Molidone (Moban), Loxapine (Loxitane), Prochlorperazine (Compazine), Trifluoperazine (Stelazine), Perphenazine (Trilafon), and Metaclopramide (Reglan).

Haloperidol has a molecular formula of C₂₁H₂₃ClFNO₂ and a molecular weight of 375.8696 g/mol. Haloperidol is also referred to as Haldol; 4-[4-(p-chlorophenyl)-4-hydroxypiperidino]-4′-fluorobutyrophenone; gamma-(4-(para-Chlorophenyl)-4-hydroxypiperidino)-para′-fluorobutyrophenone; and Serenace.

Chlorpromazine hydrochloride, a phenothiazine derivative, has a chemical formula of 2-chloro-10-[3(-dimethylamino)propyl]phenothiazine monohydrochloride. Chlorpromazine hydrochloride has the molecular formula: C₁₇H₁₉ClN₂S.HCl and a molecular weight of 355.33.

SEROQUEL® (quetiapine fumarate) is an antipsychotic drug belonging to a new chemical class, the dibenzothiazepine derivatives. The chemical designation of quetiapine fumarate is 2-[2-(4-dibenzo[b,f][1,4]thiazepin-11-yl-1-piperazinyl)ethoxy]-ethanol fumarate (2:1) (salt). Quetiapine fumarate is present in tablets as the fumarate salt. All doses and tablet strengths are expressed as milligrams of base, not as fumarate salt. Quetiapine fumarate has a molecular formula of C₄₂H₅₀N₆O₄S₂.C₄H₄O₄ and a molecular weight of 883.11 (fumarate salt).

The chemical name for clozapine is 8-chloro-11-(4-methyl-1-piperazinyl)-5H-dibenzo [b,e][1,4]diazepine. Clozapine is a an atypical antipsychotic drug which is a tricyclic dibenzodiazepine derivative. Clozapine is sold under the trademark “CLOZARIL®”. Clozapine has a molecular weight of 326.83 and a molecular formula of C₁₈H₁₉ClN₄.

The chemical name of trifluoperazine hydrochloride is 10-[3-(4-methyl-1-piperazinyl) propyl]-2-(trifluoromethyl)phenothiazine dihydrochloride. Trifluoperazine has a molecular weight of 480.43 and a molecular formula of C₂₁H₂₄F₃N₃S.2HCl.

Metoclopramide hydrochloride is a white crystalline, odorless substance, freely soluble in water. The chemical name of metoclopramide is 4-amino-5-chloro-N-[2-(diethylamino)ethyl]-2-methoxy benzamide monohydrochloride monohydrate. Metoclopramide has a molecular weight of 354.3.

Fluphenazine hydrochloride is a trifluoro-methyl phenothiazine derivative intended for the management of schizophrenia The chemical name of fluphenazine is 4-[3-[2-(Trifluoro-methyl)phenothiazin-10-yl]propyl]-1-piperazineethanol dihydrochloride. The molecular formular of fluphenazine is C₂₂H₂₆F₃N₃OS.2HCl and its molecular weight is 510.44.

Compositions according to the first, second, third or fourth aspects of the invention may take a number of different forms depending, in particular on the manner in which the composition is to be used. Thus, for example, the composition may be in the form of a powder, tablet, capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given and enables delivery of the compounds to the brain.

The composition of the invention may be used in a number of ways. For instance, systemic administration may be required in which case the compound may be contained within a composition which may, for example, be ingested orally in the form of a tablet, capsule or liquid. Alternatively, the composition may be administered by injection into the blood stream. Injections may be intravenous (bolus or infusion) or subcutaneous (bolus or infusion). The compounds may also be administered by inhalation (e.g. intranasally).

Compounds as defined by general formula (I) may also be administered centrally by means of intracerebral, intracerebroventricular, or intrathecal delivery.

The compositions are particularly useful when incorporated into patches that may be applied to the skin for transdermal delivery of the compounds according to general formula (I).

The compound may also be incorporated within a slow or delayed release device. Such devices may, for example, be inserted on or under the skin and the compound may be released over weeks or even months. Such a device may be particularly useful for patients with long-term dyskinesia such as patients on continuous L-DOPA therapy for the treatment of Parkinsonism. The devices may be particularly advantageous when a compound is used which would normally require frequent administration (e.g. at least daily ingestion of a tablet or daily injection).

It will be appreciated that the amount of a compound required is determined by biological activity and bioavailability which in turn depends on the mode of administration, the physicochemical properties of the compound employed and whether the compound is being used as a monotherapy or in a combined therapy. The frequency of administration will also be influenced by the abovementioned factors and particularly the half-life of the compound within the subject being treated.

Optimal dosages to be administered may be determined by those skilled in the art, and will vary with the particular compound in use, the strength of the preparation, the mode of administration, and the advancement of the disease condition. Additional factors depending on the particular subject being treated will result in a need to adjust dosages, including subject age, weight, gender, diet, and time of administration.

Known procedures, such as those conventionally employed by the pharmaceutical industry (e.g. in vivo experimentation, clinical trials etc), may be used to establish specific formulations of compositions and precise therapeutic regimes (such as daily doses of the compounds and the frequency of administration).

Generally, a daily dose of between 0.01 μg/kg of body weight and 1.0 g/kg of body weight of a compound of general formula (I) may be used for the treatment of dyskinesia depending upon which specific compound is used, more preferably, the daily dose is between 0.01 mg/kg of body weight and 100 mg/kg of body weight.

Purely by way of example a suitable dose of tofisopam for treating L-DOPA induced dyskinesia in subjects with Parkinson's disease is between 0.1 mg/kg/day and 100 mg/kg/day (depending upon the health status of the individual). It is preferred that between 0.25 mg/kg/day and 20 mg/kg/day of tofisopam is given to a person daily and is most preferred that about 10 mg/kg/day or 15 mg/kg/day tofisopam is given for treating dyskinesia induced by L-DOPA.

It will be appreciated that the required dose will be influenced by the route of administration. When tofisopam is given intravenously 0.1-10 mg/kg is a preferred dose whereas higher doses (e.g. 5-15 mg/kg) may be a suitable dose orally.

By way of further example suitable doses of Girisopam or Nerisopam are preferably 0.5-30 mg/kg.

Daily doses may be given as a single administration (e.g. a daily tablet for oral consumption or as a single daily injection). Alternatively the compound used may require administration twice or more times during a day. As an example, tofisopam for treating L-DOPA induced dyskinesia in patients with Parkinson's disease may be administered as two (or more depending upon the severity of the dyskinesia) daily doses of between 25 mg and 5000 mg in tablet form. A patient receiving treatment may take a first dose upon waling and then a second dose in the evening (if on a two dose regime) or at 3 or 4 hourly intervals thereafter. Alternatively a slow release device may be used to provide optimal doses to a patient without the need to administer repeated doses.

This invention further provides a pharmaceutical composition comprising a therapeutically effective amount of the compound of the invention and a pharmaceutically acceptable vehicle. In one embodiment, the amount of the compound (e.g. tofisopam) is an amount from about 0.01 mg to about 800 mg. In another embodiment, the amount is from about 0.01 mg to about 500 mg. When the compound is tofisopam, the amount of tofisopam may be an amount from about 0.01 mg to about 250 mg; preferably about 0.1 mg to about 60 mg; and more preferably about 1 mg to about 20 mg.

In a further embodiment, the vehicle is a liquid and the composition is a solution. In another embodiment, the vehicle is a solid and the composition is a tablet. In a further embodiment, the vehicle is a gel and the composition is a suppository.

This invention provides a pharmaceutical composition made by combining a therapeutically effective amount of a compound of general formula I and a pharmaceutically acceptable vehicle.

Compounds of general formula I are preferably combined with a pharmaceutically acceptable vehicle prior to administration.

This invention provides a process for making a pharmaceutical composition comprising combining a therapeutically effective amount of a compound of general formula I and a pharmaceutically acceptable vehicle.

In the subject invention a “therapeutically effective amount” is any amount of a compound or composition which, when administered to a subject suffering from a disease against which the compounds are effective, causes reduction, remission, or regression of the disease. A “subject” is a vertebrate, mammal, domestic animal or human being.

In the practice of this invention the “pharmaceutically acceptable vehicle” is any physiological vehicle known to those of ordinary skill in the art useful in formulating pharmaceutical compositions.

In one embodiment, the pharmaceutical vehicle may be a liquid and the pharmaceutical composition would be in the form of a solution. In another embodiment, the pharmaceutically acceptable vehicle is a solid and the composition is in the form of a powder or tablet. In a further embodiment, the pharmaceutical vehicle is a gel and the composition is in the form of a suppository or cream. In a further embodiment the compound or composition may be formulated as a part of a pharmaceutically acceptable transdermal patch.

A solid vehicle can include one or more substances which may also act as flavoring agents, lubricants, solubilizers, suspending agents, fillers, glidants, compression aids, binders or tablet-disintegrating agents; it can also be an encapsulating material. In powders, the vehicle is a finely divided solid which is in admixture with the finely divided active ingredient. In tablets, the active ingredient is mixed with a vehicle having the necessary compression properties in suitable proportions and compacted in the shape and size desired. The powders and tablets preferably contain up to 99% of the active ingredient. Suitable solid vehicles include, for example, calcium phosphate, magnesium stearate, talc, sugars, lactose, dextrin, starch, gelatin, cellulose, polyvinylpyrrolidine, low melting waxes and ion exchange resins.

Liquid vehicles are used in preparing solutions, suspensions, emulsions, syrups, elixirs and pressurized compositions. The active ingredient can be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellent.

Liquid pharmaceutical compositions which are sterile solutions or suspensions can be utilized by for example, intramuscular, intrathecal, epidural, intraperitoneal or subcutaneous injection. Sterile solutions can also be administered intravenously. The compounds may be prepared as a sterile solid composition which may be dissolved or suspended at the time of administration using sterile water, saline, or other appropriate sterile injectable medium. Vehicles are intended to include necessary and inert binders, suspending agents, lubricants, flavorants, sweeteners, preservatives, dyes, and coatings.

The compounds of general formula I can be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like.

A compound of general formula I can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

The compounds may be combined with a pharmaceutically acceptable vehicle and another therapeutically active agent prior to administration. The other therapeutically active agent may be for the treatment of parkinsonism (including Parkinson's disease).

In another embodiment of the present invention, the compound may combined with a pharmaceutically acceptable vehicle and another therapeutically active agent, wherein such agent is an antipsychotic agent used for the treatment of psychoses, prior to administration.

An embodiment of the present invention will now be described, by way of example, with reference to the accompanying drawings, in which;

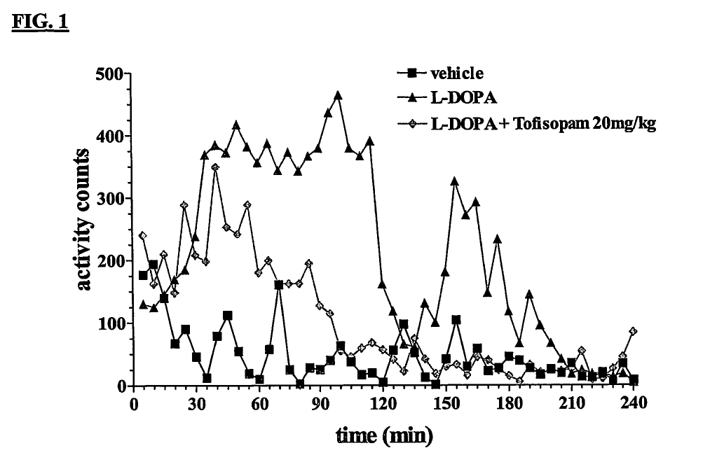

FIG. 1 is a graph illustrating the effect of Tofisopam on motor activity induced by L-DOPA in parkinsonian (MPTP-lesioned) marmosets in Example 1;

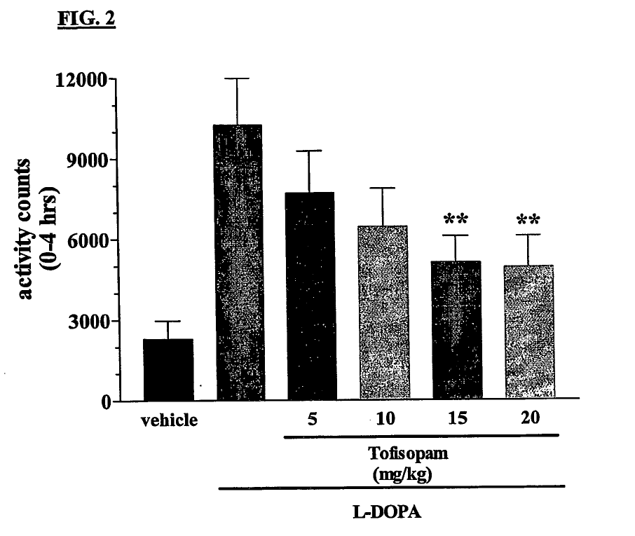

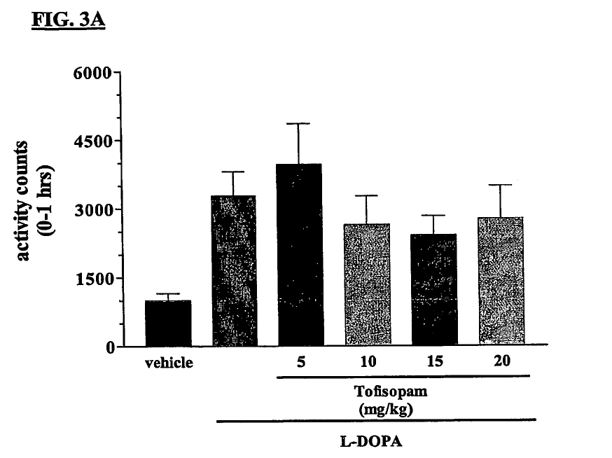

FIG. 2 is a bar chart illustrating the dose/response relationship of the effect of Tofisopam on motor activity induced by L-DOPA, cumulated over a 4 hour observation period, in parkinsonian (MPTP-lesioned) marmosets of Example 1;

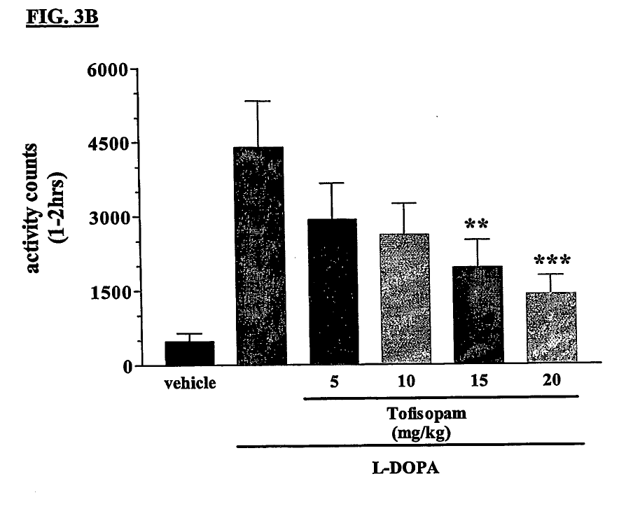

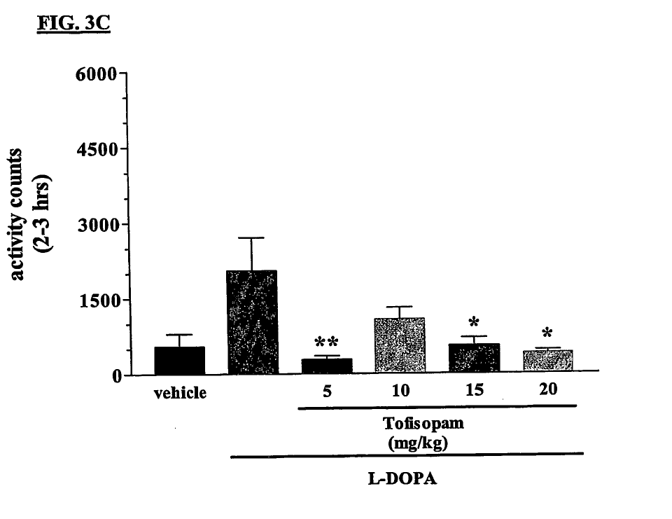

FIG. 3 is a series of bar charts (A-D) illustrating the dose/response relationship of the effect of Tofisopam on activity induced by L-DOPA, broken down into 1 hour time bins, in parkinsonian (MPTP-lesioned) marmosets of Example 1;

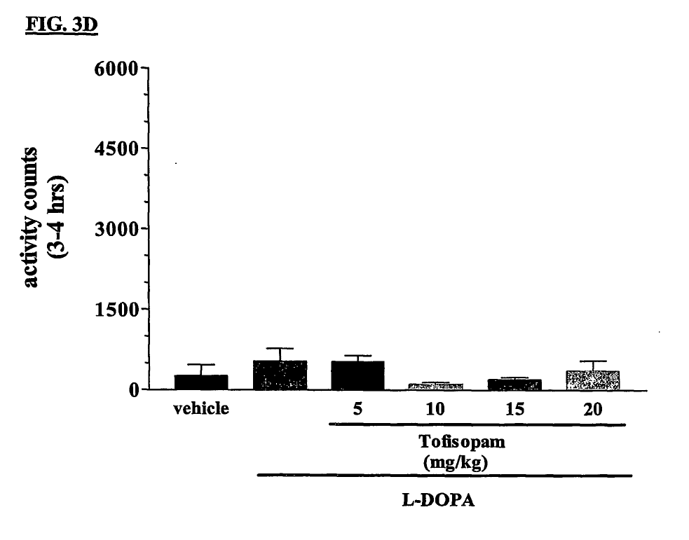

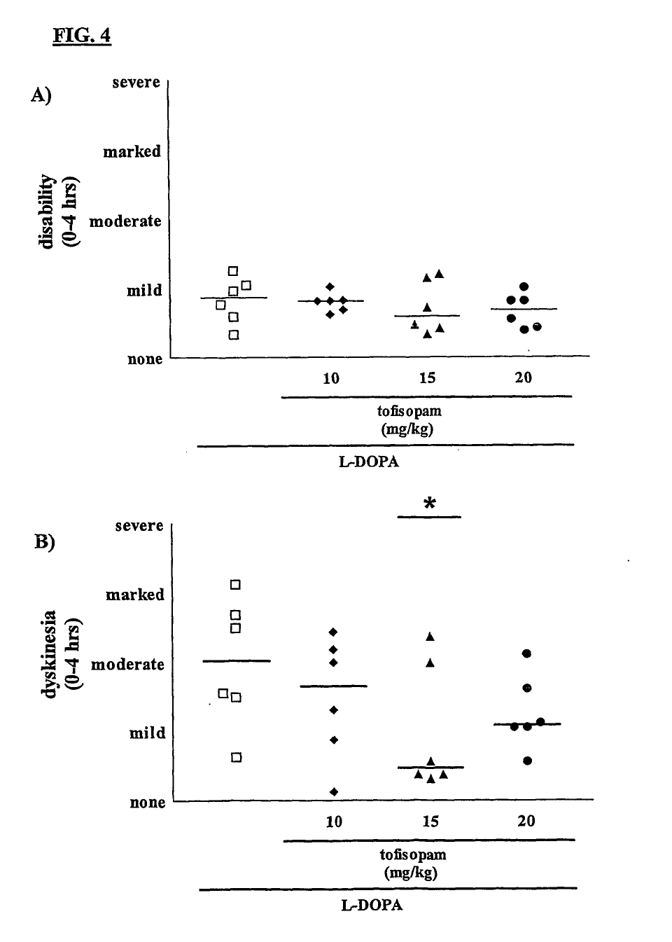

FIG. 4 is a graph illustrating the effect of tofisopam (10-20 mg/kg) on L-DOPA induced dyskinesia (A) and parkinsonian disability (B) in parkinsonian (MPTP-lesioned) marmosets of Example 1; and

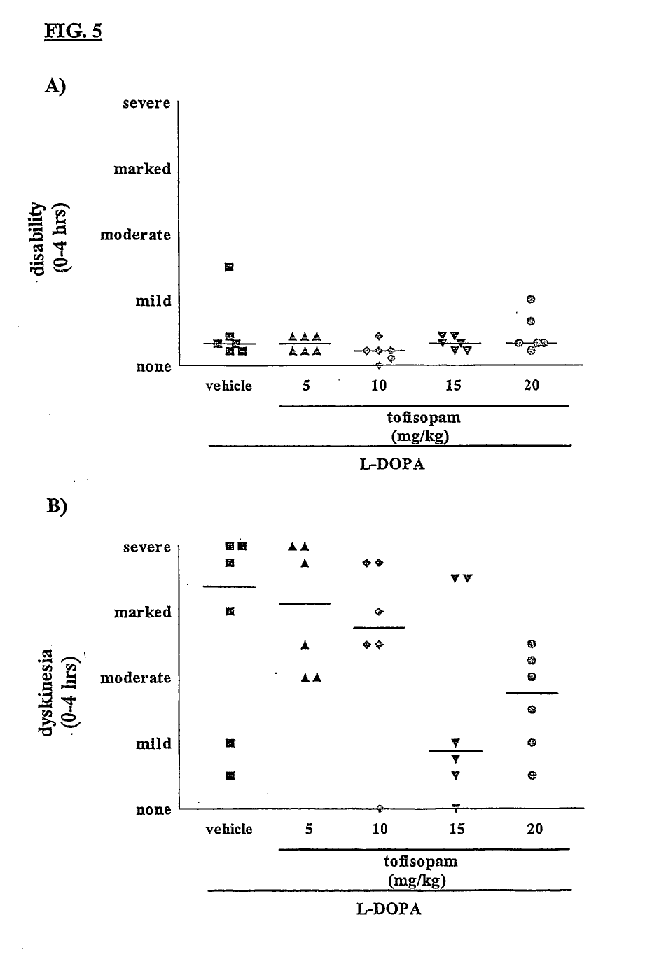

FIG. 5 is a graph illustrating the effect of tofisopam (5-20 mg/kg) on IL-DOPA induced dyskinesia (A) and parkinsonian disability (B) in parkinsonian (MPTP-lesioned) marmosets of Example 1.

For all figures: *indicates P<0.05; **indicates P<0.01; and ***indicates P<0.001 compared to L-DOPA+vehicle; non-parametric one-way repeated measures ANOVA (Friedman test) followed by Dunn's multiple comparison test.

### EXAMPLE 1

The effect of Tofisopam on L-DOPA-induced dyskinesia was assessed in the MPTP-lesioned marmoset model of Parkinson's disease.

1.1. Methods

1.1.1 Preparation of MPTP-lesioned marmoset model of Parkinson's disease Marmosets (Callithrix jacchus) (bred in a closed colony at the University of Manchester) were rendered parkinsonian by subcutaneous injection of 2 mg kgl MPTP for 5 consecutive days. The marmosets were allowed to recover for a minimum of 10 weeks until their parkinsonism became stable. The degree of activity and disability before and after MPTP treatment was assessed using a combination of scales as described in section 1.1.2. Animals were then treated with L-DOPA for at least 3 weeks to prime them to elicit dyskinesia.

1.1.2 Assessment of Behaviour

Behaviour was Assessed using the Following Scales:

(a) Activity—a measure of the motor activity of the animals that is assessed by passive infra-red sensors every five minutes. This measure assesses all movements of the animal including dyskinesia.

(b) Parkinsonian disability—non-parametric measures based on the following scales:

Mobility score: 0=no movement, 1=movement of head on the floor of the cage, 2=movement of limbs, but no locomotion, on the floor of the cage, 3=movement of head or trunk on wall of cage or perch, 4=movement of limbs, but no locomotion, on wall of cage or perch, 5=walking around floor of cage or eating from hopper on floor, 6=hopping on floor of cage, 7=climbing onto wall of cage or perch, 8=climbing up and down the walls of the cage or along perch, 9=running, jumping, climbing between cage walls/perch/roof, uses limbs through a wide range of motion and activity.

(c) Dyskinesia—non-parametric measures based on the following scale:

Dyskinesia score: 0=Absent, 1=Mild, fleeting, 2=Moderate, not interfering with normal activity, 3=Marked, at times interfering with normal activity, 4=Severe, continuous, replacing normal activity.

The behavioural tests were assessed every 30 minutes for 4 hours, by post hoc analysis of video-recordings by an observer blinded to the treatment.

1.1.3 Treatments

Marmosets received all treatments as described in Table 1. The treatments were randomised such that on each day all marmosets received one of the treatments. There was at least 48 hours washout between treatments.

TABLE 1

| Treatment | | Route of |
|---|---|---|
| Number | Treatment | administration |
| 1 | vehicle | oral |
| 2 | L-DOPA (15-17.5 mg/kg) | oral |
| 3 | L-DOPA (15-17.5 mg/kg) + | oral |
| | Tofisopam (5 mg/kg) | |
| 4 | L-DOPA (15-17.5 mg/kg) + | oral |
| | Tofisopam (10 mg/kg) | |
| 5 | L-DOPA (15-17.5 mg/kg) + | oral |
| | Tofisopam (15 mg/kg) | |
| 6 | L-DOPA (15-17.5 mg/kg) + | oral |
| | Tofisopam (20 mg/kg) | |

1.2. Results

FIGS. 1, 2 and 3 illustrate the effect of Tofisopam treatment on L-DOPA-induced motor counts in the MPTP-lesioned marmoset model of Parkinson's disease (see method 1.1.2a).

FIGS. 4 and 5 illustrate the results of two experiments. The figures demonstrate that Tofisopam (5-20 mg/kg) reduces the severity of L-DOPA-induced dyskinesia (see method 1.1.2c) without affecting the antiparkinsonian action (see method 1.1.2b) of L-DOPA.

These data demonstrate that compound as defined by general formula (1) cause a dose-dependent reduction in the severity of L-DOPA-induced dyskinesia. FIGS. 4 and 5, in particular, illustrate that the compounds have the benefit of reducing dyskinesia without adversely affecting the antiparkinsonian action of L-DOPA. Accordingly compounds according to the invention are particularly useful for modulating dyskinesia and, unlike prior art benzodiazepine compounds, which have been suggested to be useful for treating parkinsonism per se.

The MPTP-lesioned primate is the ‘gold standard’ preclinical model of Parkinson's disease. Therefore, these data are highly predictive of a beneficial therapeutic effect of the compounds in the treatment of L-DOPA-induced dyskinesia in Parkinson's disease patients and other dyskinesias.

1-25. (canceled)

26. A method of treating dyskinesia in a subject comprising administering to the subject a therapeutically effective amount of a compound of the formula (I): 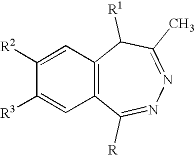
wherein R is an aryl group selected from phenyl or benzyl, which is optionally substituted with a C₁₋₆ alkyl, C₁₋₆ alkoxy, halogen, hydroxyl, amino, nitro, amido, nitrile or a carboxyl group;
R¹ is C₁₋₆ alkyl or hydrogen;
R² is C₁₋₆ alkoxy, hydrogen, hydroxyl, or halogen; and
R³ is C¹⁻⁶ alkoxy, hydrogen, hydroxyl, or halogen.

27. The method of claim 26, wherein R is selected from the following groups:

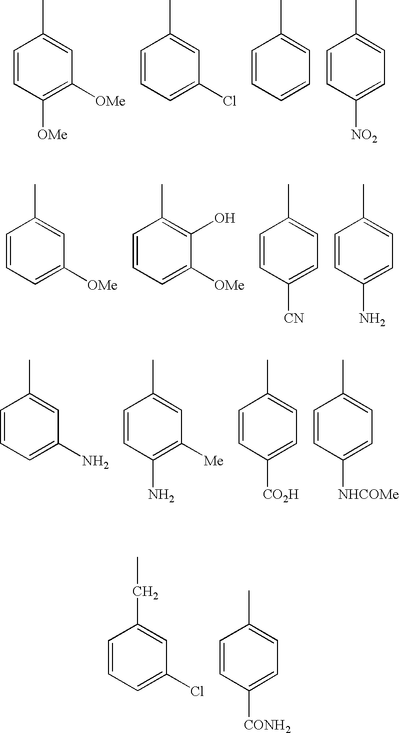

28. The method of claim 26, wherein when R¹ is an alkyl group it is C₂ alkyl(ethyl).

29. The method of claim 26, wherein when R² is an alkoxy group, it is C₁ alkoxy (methoxy).

30. The method of claim 26, wherein when R³ is an alkoxy group, it is C₁ alkoxy (methoxy).

31. The method of claim 26, wherein the compound of formula I is selected from the group comprising Tofisopam, Girisopam and Nerisoparn as shown below:

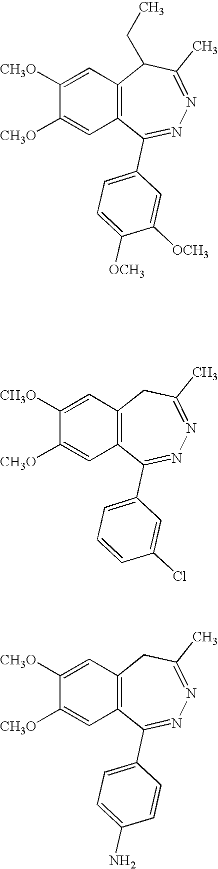

32. The method of claim 31, wherein the compound of formula I is Tofisopam.

33. The method of claim 26, wherein the compound is used for the treatment of dyskinesia associated with movement disorders.

34. The method of claim 33, wherein the compound is used for the treatment of dyskinesia associated with parkinsonism.

35. The method of claim 34, wherein the parkinsonism is idiopathic Parkinson's disease or post-encephalitic parkinsonism.

36. The method of claim 34, wherein the parkinsonism results from head injury, the treatment of schizophrenia, drag intoxication or manganese poisoning.

37. The method of claim 26, wherein the compound is used for the treatment of dyskinesia associated with Huntington's disease, idiopathic torsion dystonia, or offdystonia in Parkinson's disease.

38. The method of claim 26, wherein the compound is used for the treatment of hyperkinetic disorder associated with Tourette's syndrome and ADHD.

39. The method of claim 26, wherein the compound is used for the treatment of dyskinesia which arises as a side-effect of a therapeutic agent.

40. The method of claim 39, wherein the compound is used for the treatment of dyskinesia associated with agents used to treat movement disorders.

41. The method of claim 39, wherein the agent is used to treat parkinsonism.

42. SEW) The method of claim 41, wherein the agent is a dopamine precursor.

43. SEW) The method of claim 41, wherein the agent is a dopamine receptor agonist.

44. The method of claim 41, wherein the agent in L-DOPA.

45. The method of claim 41, wherein the agent is one of Chloro-APB, apomorphine, ropinirole, pramipexole, cabergoline, bromcriptine, lisuride or pergolide.

46. The method of claim 39, wherein the agent is used to treat schizophrenia.

47. The method of claim 46, wherein the agent is a neuroleptic.

48. The method of claim 46, wherein the agent has doparaine receptor antagonist properties.

49. The method of claim 46, wherein the agent is haloperidol clozapine, fluphenazine or sulpiride.

50. The method of claim 26, wherein the compound is used for prophylactic treatment.
